(19) United States
(12) Patent Application Publication
(10) Pub. No.: US 2026/0301960 A1
(43) Pub. Date: Oct. 1, 2026

(54) SYSTEM AND METHOD FOR IDENTIFYING GENETIC DISEASE AND DISCOVERING DISEASE ASSOCIATED GENETIC VARIANTS BASED ON MULTIPLE INSTANCE LEARNING

(71) Applicant: 3BILLION, Seoul (KR)

(72) Inventors: Kyongyeul LEE, Seoul (KR); Ho Heon KIM, Seoul (KR); Joo Yeup BAEK, Seoul (KR)

(21) Appl. No.: 19/700,567

(22) Filed: Jun. 8, 2026

Related U.S. Application Data
(63) Continuation-in-part of application No. 18/495,539, filed on Oct. 26, 2023, now abandoned.

(30) Foreign Application Priority Data
Apr. 14, 2023 (KR) 10-2023-0049291

(51) Int. Cl. G16H 50/20; G16B 20/20

(52) U.S. Cl. CPC G16H 50/20; G16B 20/20

(57) ABSTRACT

The present disclosure provides a system configured to identify a genetic disease and discover a disease-associated genetic variant, the system including a multiple instance learning model unit configured to derive identification of a genetic disease of a patient and discovery of a disease-associated genetic variant together using a multiple instance learning model configured to learn instances which are genetic variant information of the patient and a bag of the instances as input data and process, as a bag label, whether a disease of the patient is a genetic disease caused by a genetic variant.

### CROSS-REFERENCE TO RELATED APPLICATION

The present application is a continuation in part of U.S. application Ser. No. 18/495,539, filed on Oct. 26, 2023, which claims the benefit of priority to Korean Patent Application No. 10-2023-0049291 filed on Apr. 14, 2023, the contents of which are incorporated herein by reference.

### FIELD OF THE DISCLOSURE

The present disclosure relates to a system for and a method of identifying a genetic disease and discovering disease-associated genetic variants such that whether a disease of a patient is a genetic disease may be determined using multiple instance learning and a disease-associated genetic variant, among several genetic variants in the patient, may be discovered.

### BACKGROUND

A genetic disease is a disease caused by a genetic variant or a chromosomal aberration. A disease-associated genetic variant means a genetic variant that causes a genetic disease.

Interpretation of human genetic variants is a process of finding one or more disease-associated genetic variants among several genetic variants.

Generally, in order to identify a disease-associated genetic variation, a method of comparing genomic information of patients with a particular disease (cases) with genomic information of general healthy population (controls) to identify a genetic variant which is found significantly more in the cases than in the controls is being used.

Recently, research has been conducted into using artificial intelligence to determine whether a patient's disease is a genetic disease or to search for a disease-associated generic variant that causes the patient's disease.

There is research conducted to identify a disease-associated genetic variant for a patient's disease with respect to each of genetic variants in the patient using single instance learning. However, since such single instance learning needs a label for each of the genetic variants (instance label), use of the single instance learning is limited in reality.

The disclosure present presents a method of simultaneously determining whether a disease of a patient is a genetic disease, and a disease-causing genetic variant among several genetic variants using a multiple instance learning (MIL) model.

### SUMMARY

The present disclosure provides a system for and a method of identifying a genetic disease and discovering disease-associated genetic variants such that whether a disease of a patient is a genetic disease, and a disease-associated genetic variant among several genetic variants in the patient are simultaneously determined using multiple instance learning.

To accomplish the above-mentioned objects, according to an aspect of the present disclosure, there is provided a system configured to identify a genetic disease and discover a disease-associated genetic variant, the system including a multiple instance learning model unit configured to derive identification of a genetic disease of a patient and discovery of a disease-associated genetic variant together using a multiple instance learning model configured to learn instances which are genetic variant information of the patient and a bag of the instances as input data and process, as a bag label, whether a disease of the patient is a genetic disease caused by a genetic variant.

The system may include an input data processing unit configured to generate attention weights which are degrees to which the instances contribute to the identification of a genetic disease of the patient using an attention mechanism, and process the input data by reflecting the attention weights for the instances.

The input data processing unit may include: a genetic variant information embedding unit configured to embed the respective instances into low-dimensional vectors with a same dimension using respective neural networks, and then, project the low-dimensional vectors onto one manifold using weight matrices and an activation function to obtain embedding vectors identical to each other; and a genetic variant information pooling unit configured to generate attention weights for the embedding vectors using the attention mechanism, and perform a pooling process of treating the embedding vectors as one vector.

The system may include a disease and associated genetic variant determination unit configured to determine that the disease of the patient is a genetic disease caused by a genetic variant when the embedding vectors are equal to or greater than a preset reference, and discover a disease-associated genetic variant that causes the disease of the patient using the attention weights for the instances.

The multiple instance learning model may be a multi-input model using input data with various vector magnitudes.

An instance label for the instances may be generated using the attention weights for the instances, and the multiple instance learning model may be retrained using the instance label.

According to another aspect of the present disclosure, there is provided a method of identifying a genetic disease and discovering a disease-associated genetic variant, the method including: processing input data such that an input data processing unit uses instances which are genetic variant information of a patient and a bag of the instances as input data, and generates attention weights for the instances using an attention mechanism to process the input data; identifying presence of a genetic disease such that a multiple instance learning model unit identifies whether a disease of the patient is a genetic disease using a multiple instance learning model; and discovering a disease-associated generic variant such that when the disease of the patient is determined to be a genetic disease, a disease and associated genetic variant determination unit discovers a disease-associated genetic variant that causes the disease of the patient using the attention weights for the instances.

The method may further include retraining such that, when the disease of the patient is determined to be a genetic disease, an instance label is generated using the attention weights for the instances, and the multiple instance learning model is retrained using the generated instance label.

A system may be configured to identify a genetic disease and discover a disease-associated genetic variant. The system may comprise: one or more processors comprising processing circuitry; and memory storing instructions, wherein the instructions, when executed by the one or more processors, cause the system to: derive identification of the genetic disease of a patient and discovery of the disease-associated genetic variant together using a multiple instance learning model configured to learn instances which are genetic variant information of the patient and a bag of the instances as input data; process, as a bag label, whether a disease of the patient is the genetic disease caused by a genetic variant; generate first attention weights for the instances which are degrees to which the instances contribute to the identification of the genetic disease of the patient using an attention mechanism; process the input data by reflecting the first attention weights for the instances; embed the respective instances into low-dimensional vectors with a same dimension using respective neural networks; project the low-dimensional vectors onto one manifold using weight matrices and an activation function to obtain embedding vectors identical to each other; generate second attention weights for the embedding vectors using the attention mechanism; perform a pooling process of treating the embedding vectors as one vector; determine that the disease of the patient is the genetic disease caused by the genetic variant when a predicted value of the bag label is equal to or greater than a preset reference; discover the disease-associated genetic variant that causes the disease of the patient using the first attention weights for the instances; generate an instance label for the instances using the first attention weights for the instances; and retrain the multiple instance learning model using the instance label.

The multiple instance learning model may be a multi-input model using the input data with various vector magnitudes. The one or more processors may comprise one or more data processing units. The instances may comprise feature vectors extracted from genetic variants. For single nucleotide variants, the feature vectors may comprise a pathogenicity score, allele frequency values from a population database, a conservation score, protein domain annotations, and a gene-level constraint metric; and for structural variants, the feature vectors comprise a variant type indicator, variant length, breakpoint coordinates, and gene overlap counts.

The respective neural networks may comprise: a first encoder neural network for single nucleotide variants comprising at least three fully connected layers with ReLU activation functions; a second encoder neural network for structural variants comprising at least four fully connected layers with LeakyReLU activation functions; and wherein each encoder neural network projects to a common embedding dimension. The attention mechanism may comprise a gated attention mechanism applied after different variant types are integrated into a common embedding vector, and variant-type-specific knowledge may not be incorporated into gating factors.

The preset reference may comprise a calibrated probability threshold determined through isotonic regression on a validation dataset to achieve a target sensitivity of at least 95%, and wherein the system further outputs a confidence interval for the determination estimated using Monte Carlo dropout. Retraining the multiple instance learning model may comprise: generating instance labels for instances with attention weights exceeding a dynamic threshold; retraining the model using a combined loss function comprising a bag-level binary cross-entropy loss and a weighted instance-level binary cross-entropy loss; decreasing the dynamic threshold in each iteration; and terminating when instance label assignments stabilize or a maximum number of iterations is reached.

Discovering the disease-associated genetic variant may comprise: computing a contribution score for each instance as a product of a corresponding first attention weight of the first attention weights, a cosine similarity between an embedding vector of the instance and a learned disease-positive prototype vector, and variant-type-specific scaling factor; and ranking the instances by contribution score and reporting top-ranked instances as candidate disease-associated variants. The system may further comprise a clinical decision support interface configured to: receive the identification of the genetic disease and the discovered disease-associated genetic variant; retrieve treatment protocol data from a treatment database based on the identified genetic disease and the discovered disease-associated genetic variant; generate a treatment recommendation comprising at least one of a pharmaceutical intervention, a gene therapy, or a surveillance protocol specific to the identified genetic disease; and transmit the treatment recommendation to an electronic health record system.

A method of identifying a genetic disease and discovering a disease-associated genetic variant may comprise: processing input data such that an input data processing unit uses instances which are genetic variant information of a patient and a bag of the instances as input data, and generating attention weights for the instances using an attention mechanism to process the input data; determining, using a multiple instance learning model, whether a disease of the patient is the genetic disease; based on determining that the disease of the patient is the genetic disease, discovering the disease-associated genetic variant that causes the disease of the patient using the attention weights for the instances; processing, as a bag label, whether the disease of the patient is a genetic disease caused by a genetic variant; generating first attention weights for the instances which are degrees to which the instances contribute to the identification of the genetic disease of the patient using the attention mechanism; processing the input data by reflecting the first attention weights for the instances; embedding the respective instances into low-dimensional vectors with a same dimension using respective neural networks; projecting the low-dimensional vectors onto one manifold using weight matrices and an activation function to obtain embedding vectors identical to each other; generating second attention weights for the embedding vectors using the attention mechanism; performing a pooling process of treating the embedding vectors as one vector; determining that the disease of the patient is the genetic disease caused by the genetic variant when a predicted value of the bag label is equal to or greater than a preset reference; discovering the disease-associated genetic variant that causes the disease of the patient using the first attention weights for the instances; generating an instance label for the instances using the first attention weights for the instances; and retraining the multiple instance learning model using the instance label.

The method may further comprise based on discovering that the disease-associated genetic variant is a single nucleotide variation in Guanidinoacetate methyltransferase (GAMT), selecting and administering a treatment to the patient, wherein the treatment comprises at least one of creatine supplementation or ornithine supplementation. The method may further comprise based on discovering that the disease-associated genetic variant is a single nucleotide variation in Tuberous Sclerosis Complex 2 (TSC2), selecting and administering a treatment to the patient, wherein the treatment is an antiepileptic drug or a mechanistic Target of Rapamycin inhibitor. The method may further comprise administering a treatment to the patient having the genetic disease caused by the genetic variant, wherein the genetic disease is associated with a genetic mutation that involves an insertion or deletion of one or more nucleotides, wherein the genetic disease is associated with ATP-Binding Cassette Transporter Sub-Family D Member 1 (ABCD1), and wherein the treatment is a Hematopoietic stem cell transplantation (HSCT). The method may further comprise based on discovering that the disease-associated genetic variant is a pathogenic variant in ATP-Binding Cassette Transporter Sub-Family D Member 1 (ABCD1) causing X-linked adrenoleukodystrophy, selecting and administering a treatment to the patient, wherein the treatment is Hematopoietic stem cell transplantation (HSCT).

### BRIEF DESCRIPTION OF THE DRAWINGS

FIG. 1 is a configuration diagram of a system configured to identify a genetic disease and discover a disease-associated genetic variant, input data, and output data according to an embodiment of the present disclosure.

FIG. 2 is a configuration diagram of the system configured to identify a genetic disease and discover a disease-associated genetic variant, input data, and output data according to an embodiment of the present disclosure.

FIG. 3 is a configuration diagram of the system configured to identify a genetic disease and discover a disease-associated genetic variant according to an embodiment of the present disclosure.

FIG. 4 is a diagram illustrating an execution process performed by the system configured to identify a genetic disease and discover a disease-associated genetic variant according to an embodiment of the present disclosure.

FIG. 5 is a diagram illustrating a result of a test on discovering a disease-associated genetic variant using the system configured to identify a genetic disease and discover a disease-associated genetic variant according to an embodiment of the present disclosure.

FIG. 6 is a flowchart of a method of identifying a genetic disease and discovering disease-associated genetic variant according to an embodiment of the present disclosure.

### DETAILED DESCRIPTION

It will be understood that terms such as “include” or “have,” when used herein, are not intended to preclude a possibility that one or more other features, numbers, steps, operations, components, parts, or combinations thereof may exist or may be added.

In this specification, the singular includes the plural unless specifically stated otherwise. For example, an instance may be described in singular, but a plurality of instances may be also included.

Some implementations and methods for identifying disease-associated genetic variants suffer from several technical limitations. Single instance learning approaches may require instance-level labels for each genetic variant, which are often unavailable in clinical settings. Various machine learning models may not effectively process genetic variant data of heterogeneous types (SNVS, CNVs) different feature vector SVs, having dimensions. Various systems lack the ability to provide calibrated probability outputs with quantified uncertainty, leading to unreliable clinical decision support. Various neural network architectures may fail to project heterogeneous variant types onto a common embedding space, preventing meaningful comparison and aggregation of different variant types. The present disclosure addresses these technical problems by providing a multi-input neural network architecture (e.g., with variant-type-specific encoders that project heterogeneous genetic variant data onto a shared manifold), enabling unified processing of SNVs, SVs, and CNVs within a single model. The attention-based pooling mechanism may eliminate the need for instance-level labels while simultaneously enabling variant-level contribution scoring. The iterative semi-supervised retraining protocol may progressively improve model performance without requiring additional labeled data. Upon discovering a disease-associated genetic variant, the system may output treatment recommendations specific to the identified genetic disease. In an aspect, when the disease-associated genetic variant is identified as a pathogenic single nucleotide variant in the GAMT gene associated with Guanidinoacetate methyltransferase deficiency (cerebral creatine deficiency syndrome type 2), the system may recommend administration of creatine supplementation (e.g., creatine monohydrate at 100-800 mg/kg/day) and/or ornithine supplementation to restore creatine levels and reduce guanidinoacetate accumulation. In another aspect, when the disease-associated genetic variant is identified as a pathogenic variant in the TSC2 gene associated with Tuberous Sclerosis Complex, the system may recommend administration of antiepileptic drugs for seizure management and/or mechanistic Target of Rapamycin (mTOR) inhibitors such as everolimus or sirolimus to address tumor growth and other manifestations. In another aspect, when the disease-associated genetic variant is identified as a pathogenic variant in the ABCD1 gene associated with X-linked adrenoleukodystrophy, the system may recommend hematopoietic stem cell transplantation (HSCT) for patients with early cerebral disease, which can halt disease progression when performed before significant neurological decline. Hereinafter, to obviate those problems, an embodiment of the present disclosure will be described in detail with reference to the accompanying drawings.

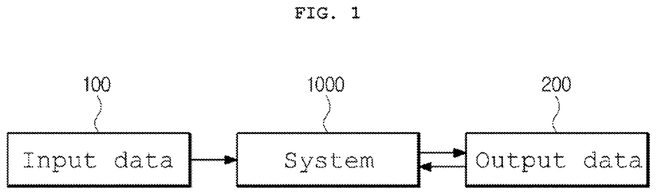

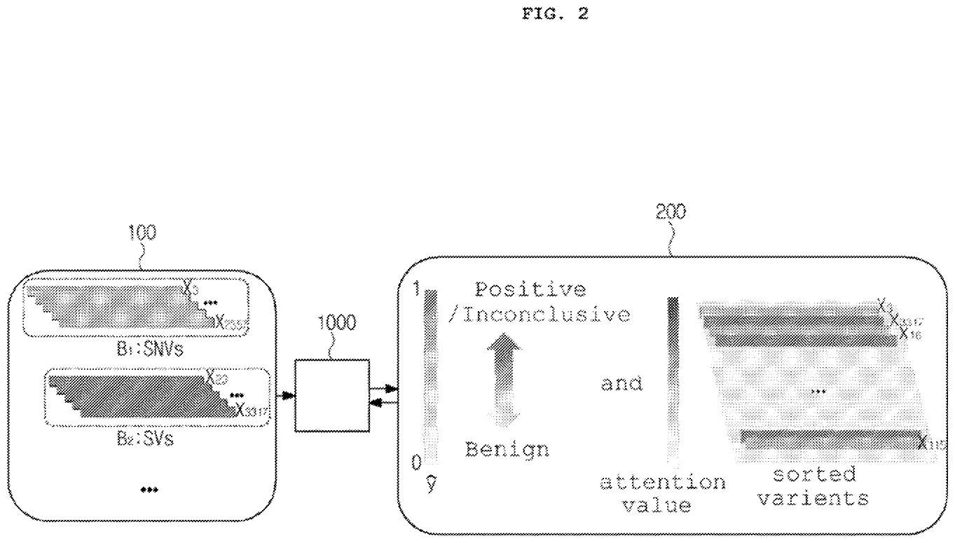

FIGS. 1 and 2 are configuration diagrams of a system 1000 configured to identify a genetic disease and discover a disease-associated genetic variant, input data, and output data according to an embodiment of the present disclosure.

Referring to FIGS. 1 and 2, the system 1000 configured to identify a genetic disease and discover a disease-associated genetic variant according to an embodiment of the present disclosure may learn input data 100 to generate output data 200.

The system 1000 configured to identify a genetic disease and discover a disease-associated genetic variant according to an embodiment of the present disclosure may use a multiple instance learning model and an attention mechanism.

The multiple instance learning model presents a type of supervised learning and provides a learning method of, when several data points (=instances) are constituted as one bundle (bag), handling a classification of the bundle (bag).

The system 1000 configured to identify a genetic disease and discover a disease-associated genetic variant according to the present disclosure handles information of each of genetic variants as an instance and learns a bag of instances as input data 100, and whether a disease of a patient is a genetic disease caused by a genetic variant is a bag label, and the bag label is handled as output data 200. In this case, the multiple instance learning model may be a multi-input model using input data with various vector magnitudes.

As an example, the input data 100 may have a genetic variant in the patient as an instance, and include a bundle (bag) of various genetic variants (e.g., a single nucleotide variant (SNV), a structural variant (SV), a copy number variant (CNV), etc.).

A single nucleotide variant (SNV) is a single base mutation, and means a substitution of one base for another base in a deoxyribonucleic acid (DNA) base sequence. For example, when C is changed to T, this is known as a C-to-T mutation or single nucleotide polymorphism (SNP).

A structural variant (SV) refers to a large structural alteration within a gene. This structural alteration usually occurs when DNA base sequences in two regions are moved, deleted, duplicated, or reversed. This structural alteration may have a great effect on the DNA base sequence.

A copy number variant (CNV) refers to a case in which a particular DNA base sequence is present in two or more copies. The CNV is also known as a genetic cause associated with a human disease.

The system 1000 configured to identify a genetic disease and discover a disease-associated genetic variant according to the present disclosure may determine whether the disease of the patient is a genetic disease caused by a genetic variant using the multiple instance learning model, and discover a disease-associated genetic variant using the attention mechanism. That is, the output data 200 may be a result of identifying whether the disease of the patient is a genetic disease caused by a genetic variant, and a disease-associated genetic variant among several genetic variants in the patient.

As an example, whether the disease of the patient is a genetic disease caused by a genetic variant (y) may be expressed as a value from 0 to 1, and when the value is equal to or greater than a preset reference value, the disease of the patient may be identified as a genetic disease caused by a genetic variant. Here, y may be referred to as a predicted value of the bag label, as the bag label indicates whether the disease of the patient is the genetic disease caused by a genetic variant.

Also, as will be described later, a disease-associated genetic variant may be identified among several genetic variants in the patient using attention weights for respective genetic variants generated using the attention mechanism.

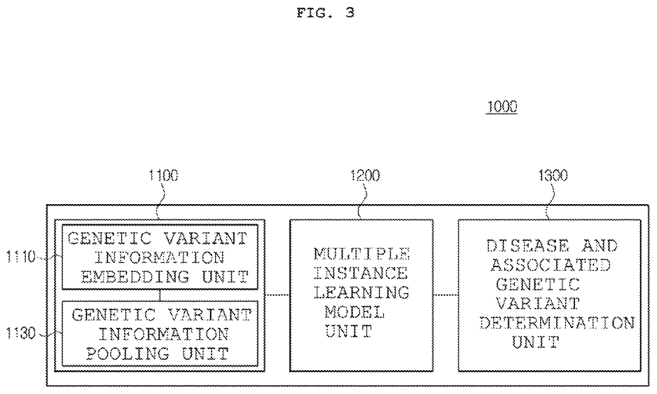

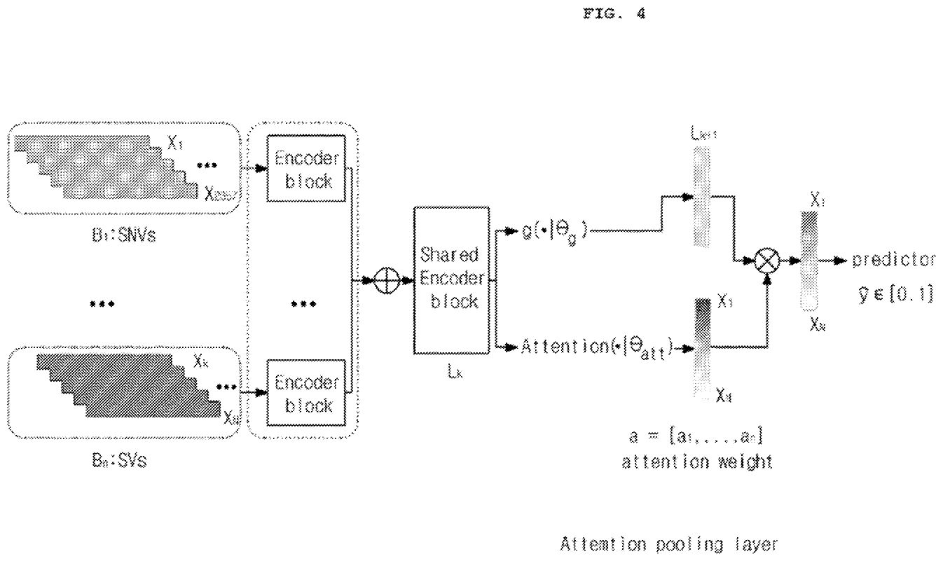

FIG. 3 is a configuration diagram of the system configured to identify a genetic disease and discover a disease-associated genetic variant according to an embodiment of the present disclosure. FIG. 4 is a diagram illustrating an execution process performed by the system configured to identify a genetic disease and discover a disease-associated genetic variant according to an embodiment of the present disclosure.

A term ‘module’ or ‘unit’ used in the specification means a software or hardware component, and the ‘module’ or ‘unit’ performs certain roles. In some embodiments, the ‘module’ or ‘unit’ is not meant to be limited to software or hardware. The ‘module’ or ‘unit’ may be configured to reside on an addressable storage medium or may be configured to reproduce one or more processors. Accordingly, as an example, the ‘module’ or ‘unit’ may include at least one of components such as software components, object-oriented software components, class components, and task components, and processes, functions, properties, procedures, subroutines, segments of program code, drivers, firmware, microcode, circuitry, data, databases, data structures, tables, arrays, or variables. Functions provided within the components and the ‘modules’ or ‘units’ may be combined into a smaller number of components and ‘modules’ or ‘units’ or may be further separated into additional components and ‘modules’ or ‘units’.

According to one or more aspects of the present disclosure, the ‘module’ or ‘unit’ may be implemented with a processor and a memory. The ‘processor’ should be broadly interpreted to include a general-purpose processor, a central processing unit (CPU), a microprocessor, a digital signal processor (DSP), a controller, a microcontroller, a state machine, and the like. In some environments, the ‘processor’ may refer to an application-specific integrated circuit (ASIC), a programmable logic device (PLD), a field programmable gate array (FPGA), and the like. The ‘processor’ may refer to a combination of processing devices such as, for example, a combination of a DSP and a microprocessor, a combination of a plurality of microprocessors, a combination of one or more microprocessors in conjunction with a DSP core, or a combination of any other such configurations. For example, the ‘memory’ should be broadly interpreted to include any electronic component capable of storing electronic information. The ‘memory’ may refer to various types of processor-readable media such as random access memory (RAM), read-only memory (ROM), non-volatile random access memory (NVRAM), programmable read-only memory (PROM), erasable programmable read-only memory (EPROM), electrically erasable PROM (EEPROM), flash memory, magnetic or optical data storage, registers, and the like. The memory is said to be in electronic communication with the processor if the processor may read information from and/or write information to the memory. The memory integrated into the processor is in electronic communication with the processor.

Referring to FIGS. 3 and 4, the system 1000 configured to identify a genetic disease and discover a disease-associated genetic variant according to an embodiment of the present disclosure includes an input data processing unit 1100, a multiple instance learning model unit 1200, and a disease and associated genetic variant determination unit 1300.

The input data processing unit 1100 may use an attention mechanism to generate an attention weight, which is a degree to which each of the instances contributes to identification of a genetic disease of the patient, and process input data by reflecting the attention weight for each of the instances.

The attention mechanism provides a processing method capable of performing learning and identification by focusing on important parts of the input data in a deep learning model. In a case of general deep learning, all parts of input data are processed with equal weight. Thus, even when a part of the input data contains important information, it was difficult to recognize important information. The attention mechanism is used to help the deep learning model to learn to find parts with importance in the input data and perform calculation by multiplying an input by a degree of the importance to allow the deep learning model to improve recognition of important information.

The input data processing unit 1100 includes a genetic variant information embedding unit 1110 and a genetic variant information pooling unit 1130. The system may be implemented on a graphics processing unit (GPU) cluster comprising a plurality of GPUs configured for parallel matrix operations. The genetic variant information embedding unit may distribute the embedding computations across multiple GPUS, with each GPU processing a subset of the genetic variants in parallel. The attention weight computations may be performed using tensor cores optimized for mixed-precision matrix multiplication. The system may be implemented on a field-programmable gate array (FPGA) configured with custom logic circuits for the attention mechanism computations. The FPGA implementation may achieve lower latency than GPU implementations for real-time clinical applications.

The genetic variant information embedding unit 1110 may embed the respective instances into low-dimensional vectors with a same dimension using each neural network, and then, project the low-dimensional vectors onto one manifold using weight matrices and an activation function to obtain same embedding vectors.

The genetic variant information pooling unit 1130 may generate attention weights for embedding vectors using the attention mechanism, and perform a pooling process of treating the embedding vectors as one vector.

For example, when two types of mutations such as an SNV and an SV are transmitted as input values, a set (bag) of variants may be defined with an expression as shown in Equation 1, and an instance ( ) may be defined with an expression as shown in Equation 2. Additionally, the SNV has f feature values, and the SV has h feature values.

$$B = \{ {B}_{snv} , {B}_{sv} \} \qquad Equation 1$$

$${B}_{snv} = \{ {x}_{1} , … , {x}_{{m}_{1}} \} \qquad Equation 2$$

$${B}_{sv} = \{ {x}_{1} , … , {x}_{{m}_{2}} \}$$

$$where {x}_{i} ∈ {ℝ}^{f} and {x}_{i}^{′} ∈ {ℝ}^{h}$$

As shown in Equations 3 and 4 below, the genetic variant information embedding unit 1110 may embed the SNV and the SV in the patient into low-dimensional vectors with a same dimension using respective neural networks, and then, project the low-dimensional vectors onto one manifold using a same weight matrix.

$${L}_{k , snv} = {f}_{snv} ( {B}_{snv} ) \qquad Equation 3$$

$${L}_{k , sv} = {f}_{sv} ( {B}_{sv} )$$

$$for {L}_{k} = {L}_{k , snv} ⊕ {L}_{k , sv} \qquad Equation 4$$

$${L}_{k + 1} = g ( {L}_{k} \vert {θ}_{g} )$$

wherein Lₖ₊₁∈ [symbol] ^{n×f}; θ_{g} is trainable parameters, embedding layer g(·); with non-linear activation function.

Here, a concatenation of a matrix or vector is denoted by ⊕, and an element wise product of the matrix_is denoted by ⊙.

The genetic variant information pooling unit 1130 may obtain attention weights for embedding vectors obtained for respective genetic variants and obtain a weighted sum of the respective embedding vectors. The obtained attention weights may be regarded as importance degrees of the respective variants, and a variant having a large value of an importance degree may be interpreted as a disease-associated generic variant.

The genetic variant information pooling unit 1130 may obtain attention weights for individual genetic variants using the attention mechanism to obtain importance degrees of embedding vectors obtained for the individual genetic variants. After passing the embedding vectors through a two-layer neural network as shown in Equation 5, the attention weights may be obtained by passing the embedding vectors through a softmax function as shown in Equation 6.

$$for {L}_{k + 1} = {[ {l}_{1} , … , {l}_{n} ]}^{T} ∈ {R}^{n \times \text{?}} , {l}_{l} is a row vector for {L}_{k + 1} \qquad Equation 5$$

$${l}_{i}^{+} = tanh ( {l}_{i} {W}_{1} ) {w}_{2}$$

$$where {W}_{1} ∈ {ℝ}^{\text{?}} , {w}_{2} ∈ {ℝ}^{\text{?}}$$

$${a}_{i} = \frac{{e}^{{l}_{i}^{+}}}{{\sum }_{j} {e}^{{l}_{j}^{+}}} \qquad Equation 6$$

$$\text{?} \text{indicates text missing or illegible when filed}$$

where e is a natural constant.

As shown in Equation 7 below, an aggregated embedding vector for a disparity axis may be obtained by calculating a dot product of respective column vectors and the obtained attention weights in a matrix with the embedding vectors obtained in Equation 4 as row vectors.

$$for a = [ {a}_{1} , … , {a}_{n} ] \qquad Equation 7$$

$$z = {aL}_{k + 1}$$

where z is f−dimensional vector

The multiple instance learning model unit 1200 may predict a degree of a possibility in which the patient may actually have a genetic disease, by passing the aggregated embedding vectors through a single-layer neural network, as shown in Equation 8 below.

$$\hat{y} = σ ( {zW}_{\text{?}} + {b}_{\text{?}} ) \qquad Equation 8$$

$$\text{?} \text{indicates text missing or illegible when filed}$$

where σ(·) is sigmoid function,
and w_{z}∈ [symbol] ^{f×1}, b_{z}∈ [symbol] ¹ are trainable parameters.

The disease and associated genetic variant determination unit 1300 may determine that the disease of the patient is a genetic disease caused by a genetic variant when the aggregated embedding vectors are equal to or greater than a preset reference, and thus, discover a disease-associated genetic variant that causes the disease of the patient by using the attention weights for the instances.

The disease and associated genetic variant determination unit 1300 may order genetic variants according to contribution values obtained by calculating a dot product of the attention weights obtained in Equation 6 and encoded genetic variant information. Then, preset high-rank genetic variants may be regarded as disease-associated generic variants.

The genetic variant information for single nucleotide variants (SNVs) may comprise a feature vector having a dimension f (e.g., f is in the range of 20 to 50 features). The SNV feature vector may include: (i) a Combined Annotation Dependent Depletion (CADD) score normalized to a range of 0 to 1; (ii) a Rare Exome Variant Ensemble Learner (REVEL) score; (iii) allele frequency values from gnomAD database for each of a plurality of population groups; (iv) a Genomic Evolutionary Rate Profiling (GERP) conservation score; (v) a PhyloP conservation score across vertebrate species; (vi) binary indicators for location within coding region, splice site region (e.g., within 10 base pairs of exon boundary), untranslated region, and intronic region; (vii) amino acid substitution indicators encoded using BLOSUM62 matrix values when applicable; (viii) protein domain annotations from Pfam database encoded as one-hot vectors; and (ix) gene-level constraint metrics including probability of loss-of-function intolerance (pLI) score and observed/expected ratio for the gene containing the variant.

The genetic variant information for structural variants (SVs) may comprise a feature vector having a dimension h (e.g., h is in the range of 30 to 80 features). The SV feature vector may include: (i) variant type encoded as a one-hot vector indicating deletion, duplication, inversion, insertion, or translocation; (ii) variant length on a logarithmic scale; (iii) breakpoint coordinates encoded relative to nearest gene boundaries; (iv) number of genes fully contained within the variant region; (v) number of genes partially overlapping the variant region; (vi) binary indicators for overlap with known pathogenic structural variant regions from databases (e.g., ClinVar and DECIPHER databases); (vii) topologically associating domain (TAD) boundary disruption indicators; (viii) enhancer and promoter overlap counts from ENCODE regulatory element annotations; and (ix) population frequency from gnomAD-SV database.

The genetic variant information for copy number variants (CNVs) may comprise a feature vector having a dimension g (e.g., g is in the range of 15 to 40 features). The CNV feature vector may include: (i) log 2 ratio value indicating copy number state; (ii) copy number state as an integer (0, 1, 2, 3, or 4+); (iii) segment length on a logarithmic scale; (iv) number of probes or bins supporting the CNV call; (v) confidence score from the CNV calling algorithm; (vi) gene content features including count of OMIM disease genes and count of genes with high pLI scores (>0.9); (vii) overlap percentage with known pathogenic CNV regions; and (viii) reciprocal overlap with benign CNV regions from the Database of Genomic Variants.

The genetic variant information embedding unit 1110 may comprise a plurality of variant-type-specific encoder neural networks, each configured to process feature vectors of a specific variant type and project them to a common embedding space. In an example, the system may comprise three encoder networks: an SNV encoder network, an SV encoder network, and a CNV encoder network.

The SNV encoder network may comprise a multi-layer perceptron having an input layer with dimension equal to the SNV feature vector dimension f, followed by a first hidden layer with a plurality of neurons (e.g., 256 neurons), a second hidden layer (e.g., with 128 neurons), and an output layer with dimension d (the common embedding dimension, e.g., 64). Each hidden layer may be followed by: (i) a batch normalization layer; (ii) a Rectified Linear Unit (ReLU) activation function defined as ReLU (x)=max (0, x); and (iii) a dropout layer with dropout probability (e.g., p=0.3 during training). The output layer may not use an activation function, producing a d-dimensional embedding vector for each SNV instance.

The SV encoder network may comprise a multi-layer perceptron having an input layer with dimension equal to the SV feature vector dimension h, followed by a first hidden layer (e.g., with 512 neurons), a second hidden layer (e.g., with 256 neurons), a third hidden layer (e.g., with 128 neurons), and an output layer with dimension d. Each hidden layer is followed by: (i) a layer normalization layer; (ii) a Leaky Rectified Linear Unit (LeakyReLU) activation function defined as LeakyReLU (x)=max (0.01×, x); and (iii) a dropout layer (e.g., with dropout probability p=0.4 during training). The deeper architecture for the SV encoder may accommodate the higher complexity and dimensionality of structural variant features.

The CNV encoder network may comprise a multi-layer perceptron having an input layer with dimension equal to the CNV feature vector dimension g, followed by a first hidden layer (e.g., with 128 neurons) and an output layer with dimension d. Each hidden layer is followed by: (i) a batch normalization layer; (ii) a Gaussian Error Linear Unit (GELU) activation function defined as GELU (x)=x·Φ(x), where Φ(x) is the cumulative distribution function of the standard normal distribution; and (iii) a dropout layer (e.g., with dropout probability p=0.2 during training). The shallower architecture for the CNV encoder reflects the lower dimensionality of CNV features.

The low-dimensional vectors output by each encoder network may be projected onto a shared manifold using a manifold projection layer. The manifold projection layer may comprise a shared weight matrix W_proj having dimensions d×d and a bias vector b_proj having dimension d. For each embedding vector e_i from any encoder, the projected embedding vector e′_i is computed as: e′_i=σ(W_proj·e_i+b_proj), where σ is a non-linear activation function (e.g., the hyperbolic tangent function tanh (x)=(e\hat{ }x−e\hat{ }(−x))/(e\hat{ }x+e\hat{ }(−x))). The use of a shared projection layer may ensure that embedding vectors from different variant types are mapped to the same manifold, enabling meaningful comparison and aggregation.

The preset reference for determining whether the disease of the patient is a genetic disease may comprise a calibrated probability threshold determined through a calibration procedure on a held-out validation dataset. The calibration procedure may ensure that the model's predicted probabilities accurately reflect the true likelihood of the disease being genetic.

The calibration procedure may comprise applying isotonic regression to the model's raw output probabilities on the validation dataset. Isotonic regression fits a non-decreasing step function that maps raw probabilities to calibrated probabilities, minimizing the mean squared error between calibrated probabilities and true labels. The calibrated threshold 0 may be determined as the calibrated probability value that achieves a target sensitivity of at least 95% for known pathogenic cases in the validation dataset while minimizing the false positive rate.

The system may comprise a confidence interval estimation processor that quantifies in uncertainty the disease classification. The confidence interval may be estimated using Monte Carlo dropout, wherein the model performs N forward passes (preferably N=50) with dropout enabled during inference. The mean and standard deviation of the N output probabilities may be computed, and a 95% confidence interval may be constructed as [mean—1.96×std, mean±1.96×std]. Cases where the confidence interval spans the calibrated threshold 0 may be flagged for clinical review.

The retraining of the multiple instance learning model may comprise an iterative semi-supervised learning protocol that progressively generates instance labels from attention weights and uses these labels to improve model performance. This protocol may address the fundamental challenge in multiple instance learning that instance-level labels are not available during initial training.

In each iteration of the retraining protocol, instance labels may be generated for instances with attention weights exceeding a dynamic threshold t. The dynamic threshold t may be initialized at the 90th percentile of attention weights across all instances in the training set. For instances with attention weights exceeding t, a positive instance label (indicating the instance is a disease-associated variant) may be assigned if the corresponding bag label is positive (indicating the patient has a genetic disease). Instances with attention weights below t are assigned a negative instance label. Instances in bags with negative bag labels may be all assigned negative instance labels regardless of attention weights.

The multiple instance learning model may be retrained using a combined loss function L_combined comprising a bag-level loss L_bag and a weighted instance-level loss L_instance: L_combined=L_bag+λ. L_instance, where λ is a weighting parameter. The bag-level loss L_bag may be the binary cross-entropy loss between the predicted bag probability and the true bag label. The instance-level loss L_instance may be the binary cross-entropy loss between the predicted instance probabilities (derived from attention weights normalized to [0,1]) and the generated instance labels. The weighting parameter λ may be initialized at 0.1 in the first iteration and increased by 0.1 in each subsequent iteration until reaching a maximum value of 0.5.

The dynamic threshold τ may be decreased by 5 percentage points in each iteration, from the 90th percentile in the first iteration to the 85th percentile in the second iteration, and so on, until reaching a minimum of the 70th percentile. This progressive threshold decay may allow the model to gradually incorporate more instances into the semi-supervised learning process. The retraining protocol may terminate when one of the following convergence criteria is met: (i) the instance label assignments stabilize, defined as less than 1% of instances changing labels between consecutive iterations; (ii) the validation set performance (measured by area under the receiver operating characteristic curve) does not improve for 3 consecutive iterations; or (iii) a maximum of 5 iterations is reached.

Within each iteration, the model may be trained using early stopping based on validation set performance with a patience of 10 epochs. Additionally, L2 regularization with a coefficient of 1e-4 is applied to all weight matrices to prevent overfitting. The learning rate is initialized at 1e-3 and reduced by a factor of 0.5 when validation loss plateaus for 5 consecutive epochs.

The disease-associated genetic variant discovery may comprise computing a contribution score C_i for each variant i. The contribution score C_i may be computed as the product of at least two of three factors: C_i=α_i·sim_i·s{t_i}, where α_i is the first attention weight for instance i, sim_i is the cosine similarity between the embedding vector of instance i and a learned disease-positive prototype vector, and s{t_i} is a variant-type-specific scaling factor.

The disease-positive prototype vector P may be a d-dimensional vector learned during training that represents the centroid of embedding vectors for instances in positive bags (bags corresponding to patients with genetic diseases). The prototype vector P may be computed as the weighted average of embedding vectors, where the weights are the attention weights: P=Σ_i(α_i·e′_i)/Σ_i α_i, averaged across all positive bags in the training set. The cosine similarity sim_i between instance embedding e′_i and prototype P may be computed as: sim_i=(e′_i·P)/(∥e′_i∥·∥P∥).

Variants may be ranked in descending order by contribution score C_i. The top-k variants (where k is a configurable parameter, with a default value of k=5) may be reported as candidate disease-associated variants. For each reported variant, the system may output: (i) the variant identifier and genomic coordinates; (ii) the contribution score C_i; (iii) the attention weight & i; (iv) the cosine similarity sim_i; (v) a confidence interval for the contribution score estimated using bootstrap resampling with 1000 iterations; and (vi) supporting evidence from external databases (e.g., ClinVar pathogenicity classifications and literature references from PubMed).

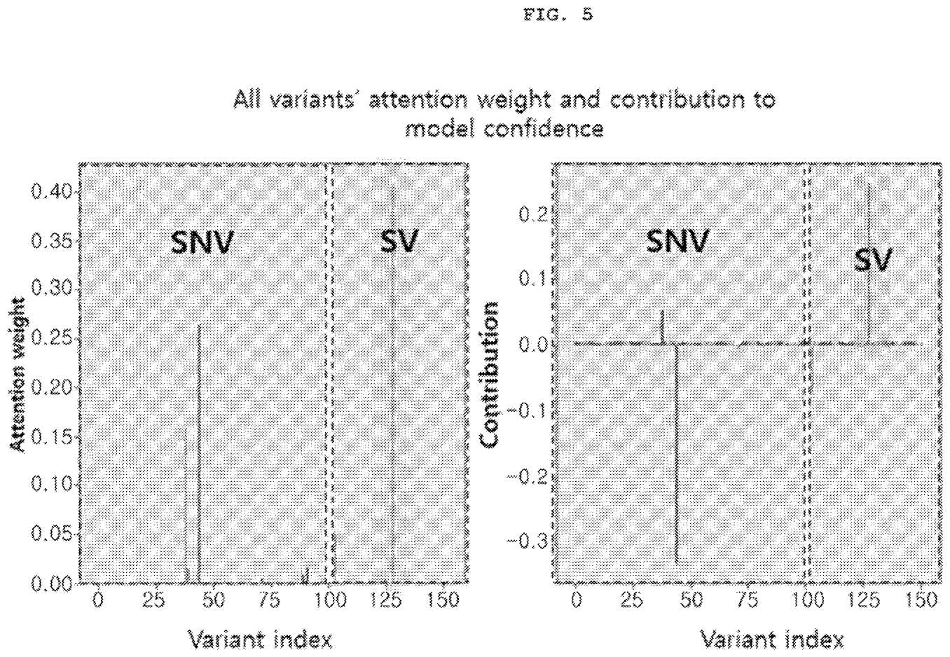

FIG. 5 is a diagram illustrating a result of a test on discovering a disease-associated genetic variant using the system configured to identify a genetic disease and discover a disease-associated genetic variant according to an embodiment of the present disclosure.

FIG. 5 illustrates identification of a genetic disease of a patient with a total of 157 variants including 100 SNVs and 57 SVs using the system configured to identify a genetic disease and discover a disease-associated genetic variant according to the present disclosure, and visualization of contributions according to each variant.

In FIG. 5, points 0 to 99 in an x-axis represent SNV indices and points 101 to 157 represent SV indices. Whether genetic disease is present is identified with a model confidence of 0.6, and it may be interpreted that a 128th variant among the SNVs and SVs contributes most. Since the system configured to identify a genetic disease and discover a disease-associated genetic variant according to the present disclosure obtains a value of 0.6 by focusing on the 128th variant with respect to a disease-associated generic variant, the 128th variant may be interpreted as a disease-associated genetic variant.

As such, the system configured to identify a genetic disease and discover a disease-associated genetic variant according to the present disclosure may determine whether a disease is a genetic disease, using the multi instance learning model without having to use a genetic variant label (instance label) of a patient.

In addition, a disease-associated generic variant in a patient may be discovered using attention weights for the patient's genetic variants generated using the attention mechanism.

That is, the system 1000 configured to identify a genetic disease and discover a disease-associated genetic variant according to the present disclosure may simultaneously determine whether a patient has a genetic disease, and a disease-associated genetic variant using the attention mechanism and the multiple instance learning model without having to use a genetic variant label (instance label) of the patient.

In addition, the system 1000 configured to identify a genetic disease and discover a disease-associated genetic variant according to the present disclosure may improve performance of the multiple instance learning model by generating a genetic variant label (instance label) for a patient using an obtained attention weight and by retraining the multiple instance learning model using the generated instance label.

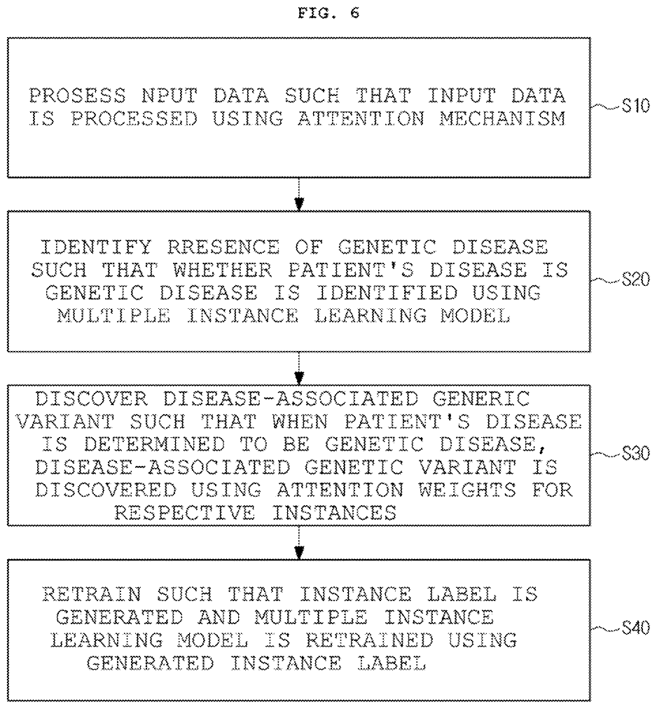

FIG. 6 is a flowchart of a method of identifying a genetic disease and discovering a disease-associated genetic variant according to an embodiment of the present disclosure.

The method of identifying a genetic disease and discovering a disease-associated genetic variant according to an embodiment of the present disclosure includes input data processing (S10), identifying presence of a genetic disease (S20), and discovering a disease-associated generic variant (S30), and a retraining (S40).

In the input data processing (S10), an input data processing unit uses instances, i.e., genetic variant information of a patient and a bag of the instances as input data, and generate attention weights for the instances using an attention mechanism to process the input data.

In the identifying of presence of a genetic disease (S20), a multiple instance learning model unit may identify whether a disease of the patient is a genetic disease using a multiple instance learning model.

In the discovering a disease-associated generic variant (S30), when the patient's disease is determined to be a genetic disease, a disease and associated genetic variant determination unit may discover a disease-associated genetic variant which causes the patient's disease using the attention weights for the instances.

In this case, when the patient's disease is not determined to be a genetic disease, the discovering of a disease-associated generic variant may not be performed.

In the retraining (S40), when the patient's disease is determined to be a genetic disease, an instance label may be generated using the attention weights for the instances, and the multiple instance learning model is retrained using the generated instance label. According to the method of identifying a genetic disease and discovering a disease-associated genetic variant according to an embodiment of the present disclosure, the multiple instance learning model may be retrained to improve performance of the multiple instance learning model.

Although the illustrative configuration of an apparatus has been shown in the present specification and the attached drawings, functional operations and implementations of the subject matter described herein may be implemented in different types of digital electronic circuits, or may be implemented in the form of computer software, firmware, or hardware including the structures disclosed herein and structural equivalents thereof or may be implemented by a combination of one or more thereof. Implementations of the subject matter described herein may include one or more computer program products, i.e., one or more modules regarding computer program instructions encoded on a tangible program storage medium to control operations of an apparatus according to the present disclosure or perform execution based on the operations. The computer-readable medium may be a machine-readable storage device, a machine-readable storage substrate, a memory device, or a combination thereof.

The present disclosure described above is not limited to the embodiments set forth herein, and it should be apparent to those skilled in the art the accompanying drawings, and various substitutions, modifications and changes may be made therein without departing from the spirit and scope of the present disclosure.

The present disclosure may determine whether a patient has a genetic disease without having to have a genetic variant label (instance label) using a multiple instance learning model.

In addition, in the present disclosure, a disease-associated generic variant in a patient may be discovered using attention weights for the patient's genetic variants generated using an attention mechanism.

In addition, in the present disclosure, whether a patient has a genetic disease and a disease-associated genetic variant may be simultaneously determined using the attention mechanism and a multiple instance learning model without having to have a genetic variant label (instance label) of the patient.

In addition, in the present disclosure, performance of the multiple instance learning model may be improved by generating the patient's genetic variant label (instance label) using the attention weights and retraining the multiple instance learning model using the generated instance label.

1. A system configured to identify a genetic disease and discover a disease-associated genetic variant, the system comprising:
one or more processors comprising processing circuitry; and
memory storing instructions,
wherein the instructions, when executed by the one or more processors, cause the system to:
derive identification of the genetic disease of a patient and discovery of the disease-associated genetic variant together using a multiple instance learning model configured to learn instances which are genetic variant information of the patient and a bag of the instances as input data;
process, as a bag label, whether a disease of the patient is the genetic disease caused by a genetic variant;
generate first attention weights for the instances which are degrees to which the instances contribute to the identification of the genetic disease of the patient using an attention mechanism;
process the input data by reflecting the first attention weights for the instances;
embed the respective instances into low-dimensional vectors with a same dimension using respective neural networks;
project the low-dimensional vectors onto one manifold using weight matrices and an activation function to obtain embedding vectors identical to each other;
generate second attention weights for the embedding vectors using the attention mechanism;
perform a pooling process of treating the embedding vectors as one vector;
determine that the disease of the patient is the genetic disease caused by the genetic variant when a predicted value of the bag label is equal to or greater than a preset reference;
discover the disease-associated genetic variant that causes the disease of the patient using the first attention weights for the instances;
generate an instance label for the instances using the first attention weights for the instances; and
retrain the multiple instance learning model using the instance label.

2. The system of claim 1, wherein the multiple instance learning model is a multi-input model using the input data with various vector magnitudes.

3. The system of claim 1, wherein the one or more processors comprises one or more data processing units.

4. The system of claim 1, wherein the instances comprise feature vectors extracted from genetic variants, wherein:
for single nucleotide variants, the feature vectors comprise a pathogenicity score, allele frequency values from a population database, a conservation score, protein domain annotations, and a gene-level constraint metric; and
for structural variants, the feature vectors comprise a variant type indicator, variant length, breakpoint coordinates, and gene overlap counts.

5. The system of claim 1, wherein the respective neural networks comprise:
a first encoder neural network for single nucleotide variants comprising at least three fully connected layers with ReLU activation functions;
a second encoder neural network for structural variants comprising at least four fully connected layers with LeakyReLU activation functions; and
wherein each encoder neural network projects to a common embedding dimension.

6. The system of claim 1, wherein the attention mechanism comprises a gated attention mechanism applied after different variant types are integrated into a common embedding vector, and wherein variant-type-specific knowledge is not incorporated into gating factors.

7. The system of claim 1, wherein the preset reference comprises a calibrated probability threshold determined through isotonic regression on a validation dataset to achieve a target sensitivity of at least 95%, and wherein the system further outputs a confidence interval for the determination estimated using Monte Carlo dropout.

8. The system of claim 1, wherein retraining the multiple instance learning model comprises:
generating instance labels for instances with attention weights exceeding a dynamic threshold;
retraining the model using a combined loss function comprising a bag-level binary cross-entropy loss and a weighted instance-level binary cross-entropy loss;
decreasing the dynamic threshold in each iteration; and
terminating when instance label assignments stabilize or a maximum number of iterations is reached.

9. The system of claim 1, wherein discovering the disease-associated genetic variant comprises:
computing a contribution score for each instance as a product of a corresponding first attention weight of the first attention weights, a cosine similarity between an embedding vector of the instance and a learned disease-positive prototype vector, and a variant-type-specific scaling factor; and
ranking the instances by contribution score and reporting top-ranked instances as candidate disease-associated variants.

10. The system of claim 1, further comprising:
a clinical decision support interface configured to:
receive the identification of the genetic disease and the discovered disease-associated genetic variant;
retrieve treatment protocol data from a treatment database based on the identified genetic disease and the discovered disease-associated genetic variant;
generate a treatment recommendation comprising at least one of a pharmaceutical intervention, a gene therapy, or a surveillance protocol specific to the identified genetic disease; and
transmit the treatment recommendation to an electronic health record system.

11. A method of identifying a genetic disease and discovering a disease-associated genetic variant, the method comprising:
processing input data such that an input data processing unit uses instances which are genetic variant information of a patient and a bag of the instances as input data, and generating attention weights for the instances using an attention mechanism to process the input data;
determining, using a multiple instance learning model, whether a disease of the patient is the genetic disease;
based on determining that the disease of the patient is the genetic disease, discovering the disease-associated genetic variant that causes the disease of the patient using the attention weights for the instances;
processing, as a bag label, whether the disease of the patient is a genetic disease caused by a genetic variant;
generating first attention weights for the instances which are degrees to which the instances contribute to the identification of the genetic disease of the patient using the attention mechanism;
processing the input data by reflecting the first attention weights for the instances;
embedding the respective instances into low-dimensional vectors with a same dimension using respective neural networks;
projecting the low-dimensional vectors onto one manifold using weight matrices and an activation function to obtain embedding vectors identical to each other;
generating second attention weights for the embedding vectors using the attention mechanism;
performing a pooling process of treating the embedding vectors as one vector;
determining that the disease of the patient is the genetic disease caused by the genetic variant when a predicted value of the bag label is equal to or greater than a preset reference;
discovering the disease-associated genetic variant that causes the disease of the patient using the first attention weights for the instances;
generating an instance label for the instances using the first attention weights for the instances; and
retraining the multiple instance learning model using the instance label.

12. The method of claim 11, further comprising:
based on discovering that the disease-associated genetic variant is a single nucleotide variation in Guanidinoacetate methyltransferase (GAMT), selecting and administering a treatment to the patient, wherein the treatment comprises at least one of creatine supplementation or ornithine supplementation.

13. The method of claim 11, further comprising:
based on discovering that the disease-associated genetic variant is a single nucleotide variation in Tuberous Sclerosis Complex 2 (TSC2), selecting and administering a treatment to the patient, wherein the treatment is an antiepileptic drug or a mechanistic Target of Rapamycin inhibitor.

14. The method of claim 11, further comprising:
administering a treatment to the patient having the genetic disease caused by the genetic variant, wherein the genetic disease is associated with a genetic mutation that involves an insertion or deletion of one or more nucleotides, wherein the genetic disease is associated with ATP-Binding Cassette Transporter Sub-Family D Member 1 (ABCD1), and wherein the treatment is a Hematopoietic stem cell transplantation (HSCT).

15. The method of claim 11, further comprising:
based on discovering that the disease-associated genetic variant is a pathogenic variant in ATP-Binding Cassette Transporter Sub-Family D Member 1 (ABCD1) causing X-linked adrenoleukodystrophy, selecting and administering a treatment to the patient, wherein the treatment is Hematopoietic stem cell transplantation (HSCT).
